(19) United States
(12) Patent Application Publication
(10) Pub. No.: US 2025/0256081 A1
(43) Pub. Date: Aug. 14, 2025

(54) MICRONEEDLE PARTICLES AND METHOD FOR PREPARING MICRONEEDLE PARTICLES

(71) Applicant: RAPHAS CO., LTD., Seoul (KR)

(72) Inventors: Seong-Jun KIM, Gimpo-si, Gyeonggi-do (KR); Sung-Su KIM, Anyang-si, Gyeonggi-do (KR); Sookie LA, Seoul (KR); Na-Young YOO, Incheon (KR); Do-Hyeon JEONG, Seoul (KR)

(73) Assignee: RAPHAS CO., LTD., Seoul (KR)

(21) Appl. No.: 18/850,065

(22) PCT Filed: Mar. 27, 2023
(86) PCT No.: PCT/KR2023/004055

§ 371 Date: Sep. 24, 2024

(30) Foreign Application Priority Data
Mar. 28, 2022 (KR) 10-2022-0037940
Nov. 29, 2022 (KR) 10-2022-0162324

(51) Int. Cl. A61M 37/00; A61K 8/02

(52) U.S. Cl. CPC A61M 37/0015; A61K 8/02; A61K 2800/87; A61M 2037/0053; A61M 2037/0061

(57) ABSTRACT

The present disclosure relates to microneedle particles and a method for preparing microneedle particles.

### TECHNICAL FIELD

The present disclosure relates to microneedle particles and a method of preparing microneedle particles.

### BACKGROUND ART

Drugs and bioactive substances are generally administered orally in tablet or capsule form, but many drugs may not be effectively delivered using this administration method alone for reasons such as digestion or absorption in the gastrointestinal tract or loss through liver mechanisms. Some drugs may not diffuse effectively across the intestinal mucosa. Patient compliance is also an issue.

Another common technique for delivering drugs and bioactive substances is to use a conventional needle. Although this method is more effective than oral administration, it has disadvantages of causing pain at the injection site, local damage to the skin, bleeding, and disease infection at the injection site.

To resolve the problems of the oral administration and subcutaneous injection, a transdermal administration method using a patch is used. Transdermal administration using patches has fewer side effects and higher patient compliance, and makes it easy to maintain a constant blood drug concentration.

As one of the transdermal administration methods described above, various microstructures including microneedles have been developed. Metals and various polymer materials have been used as materials for microneedles. Recently, biodegradable polymer materials have attracted attention as materials for microneedles.

These microneedles are manufactured in the form of patches including adhesive sheets and are used by attaching the microneedles to a desired area of the body by using the adhesive sheet.

However, these microneedle products are manufactured in the form of patches and may only be used in limited areas of the human body. Furthermore, when the adhesion of the adhesive sheet is weak, the effectiveness is very low.

### DISCLOSURE

### Technical Problem

The present disclosure is to provide microneedle particles that may be used over a wide area of the human body and may be used regardless of adhesion when used with cosmetics and the like.

### Technical Solution

An object of the present disclosure is achieved by a method of preparing microneedle particles, the method including providing a particle assembly having a plurality of microneedle particles on an upper surface of a first film, and separating the microneedle particles from the particle assembly, wherein the separating of the microneedle particles from the particle assembly includes weakening adhesion between the first film and the microneedle particles.

Here, the first film may include a water-soluble film. The first film may include one or a mixture of two or more selected from polyvinylpyrrolidone, polyvinyl alcohol, cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibrillatedcellulose, psyllium seed gum, xanthan gum, arabino galactan, gum arabic, alginic acid, gelatin, gellan gum, carrageenan, karaya gum, curdlan, chitosan, chitin, tara gum, tamarind gum, tragacanth gum, furcelleran, pectin or pullulan, hydroxypropyl methylcellulose (HPMC), hydroxyalkyl cellulose, ethyl hydroxyethyl cellulose, alkyl cellulose, gluten, soy polysaccharides, polacrilin potassium, sodium starch glycolate, crospovidone, croscarmellose sodium, bentonite, hydroxypropyl starch, sodium carboxymethyl cellulose, sodium alginate, sodium lauryl sulfate, silicic anhydride, hydroxypropyl cellulose, polyvinyl acetate, amylose, sodium hydrogen carbonate, amylopectin, sodium polyphosphate, and aluminium magnesium silicate.

The particle assembly may further include a second film attached to a lower surface of the first film.

The second film may include a hydrophobic film or a hydrophilic film. For example, the second film may include one or a mixture of two or more selected from high density polyethylene (HDPE), polystyrene (PS), polypropylene (PP), polyethylene terephthalate (PET), and polyethylene naphthalate (PEN).

Adhesion between the first film and the microneedle particles may be higher than adhesion between the second film and the microneedle particles.

The weakening of the adhesion between the first film and the microneedle particles may include treating the particle assembly with a solvent to dissolve or swell the first film.

In this case, the solvent may not dissolve the microneedle particles.

For example, the solvent may include one or more substances selected from distilled water, ethanol, acetone, oleyl alcohol, isoparaffin, and dipropylene glycol.

The method may further include, when the first film is swelled with the solvent, applying a transverse force to the microneedle particles to separate the microneedle particles from the first film.

The method may further include, when the first film is dissolved with the solvent, filtering and extracting the microneedle particles from the solvent, and drying the microneedle particles.

When the particle assembly is treated with the solvent, the particle assembly may be treated using the solvent in a liquid state or the solvent may be vaporized to treat the particle assembly with the solvent in a gaseous state.

An object of the present disclosure is achieved by microneedle particles prepared using the method of preparing microneedle particles described above.

### Advantageous Effects

According to the present disclosure having the above-described configuration, when the microneedle particles are used together with cosmetics and the like, the microneedle particles may be used over a wide area of the human body, and furthermore, may be used regardless of adhesion.

### DESCRIPTION OF DRAWINGS

FIG. 1 is a flowchart illustrating a method of preparing microneedle particles according to an embodiment of the present disclosure.

FIG. 2 is a diagram illustrating a particle assembly according to an embodiment of the present disclosure.

FIG. 3 is a conceptual diagram illustrating a process of treating a particle assembly with a solvent, according to another embodiment.

FIG. 4 is a diagram illustrating a process of separating microneedle particles from a particle assembly after solvent treatment.

FIG. 5 is a diagram illustrating a state in which microneedle particles are separated from a particle assembly by dissolving a first film after solvent treatment.

FIG. 6 is a diagram illustrating a particle assembly according to another embodiment of the present disclosure.

FIG. 7 is a diagram an operation of preparing a particle assembly according to another embodiment.

FIG. 8 is a conceptual diagram illustrating a process of treating a particle assembly with a solvent, according to another embodiment.

FIG. 9 is a diagram illustrating a process in which microneedle particles are separated from a particle assembly by swelling a first film after the particle assembly is treated with a solvent, according to another embodiment.

FIG. 10 is a conceptual diagram illustrating a process of separating microneedle particles from a particle assembly by dissolving a first film in the particle assembly, according to another embodiment.

FIG. 11 is a schematic diagram illustrating a device for treating a particle assembly by vaporizing and providing a solvent.

FIG. 12 is a conceptual diagram illustrating a process of using cosmetics containing microneedle particles according to the present disclosure.

### BEST MODE

Hereinafter, a method of preparing microneedle particles according to an embodiment of the present disclosure will be described in detail with reference to the drawings.

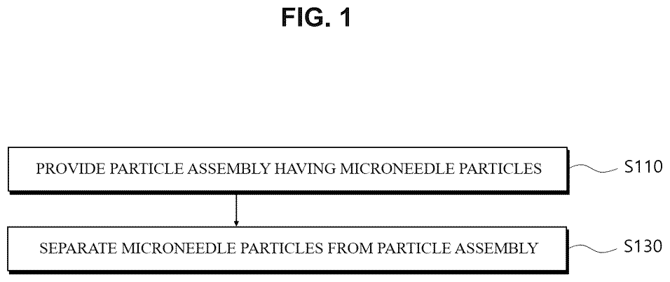

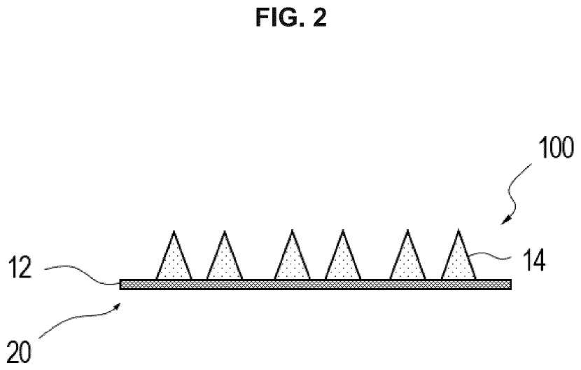

FIG. 1 is a flowchart illustrating a method of preparing microneedle particles according to an embodiment of the present disclosure. FIG. 2 is a diagram illustrating a particle assembly 100 according to an embodiment of the present disclosure.

Referring to FIGS. 1 and 2, the method of preparing microneedle particles may include providing the particle assembly 100 in which a plurality of microneedle particles 14 are formed on an upper surface of a first film 12 (S110) and separating the microneedle particles 14 from the particle assembly 100 (S130).

Here, the particle assembly 100 may be defined as a structure in which the microneedle particles 14 to be prepared according to the present disclosure are attached on a film support layer 20 including the first film 12.

For example, the particle assembly 100 may include the film support layer 20 including the first film 12 including a water-soluble film and a plurality of biodegradable microneedle particles 14 formed on the film support layer 20.

As described below, the first film 12 may include a material that is dissolved or swelled by a predetermined solvent when the microneedle particles 14 are separated from the particle assembly 100.

The first film 12 described above may include one or a mixture of two or more selected from polyvinylpyrrolidone, polyvinyl alcohol, cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibrillatedcellulose, psyllium seed gum, xanthan gum, arabino galactan, gum arabic, alginic acid, gelatin, gellan gum, carrageenan, karaya gum, curdlan, chitosan, chitin, tara gum, tamarind gum, tragacanth gum, furcelleran, pectin or pullulan, hydroxypropyl methylcellulose (HPMC), hydroxyalkyl cellulose, ethyl hydroxyethyl cellulose, alkyl cellulose, gluten, soy polysaccharides, polacrilin potassium, sodium starch glycolate, crospovidone, croscarmellose sodium, bentonite, hydroxypropyl starch, sodium carboxymethyl cellulose, sodium alginate, sodium lauryl sulfate, silicic anhydride, hydroxypropyl cellulose, polyvinyl acetate, amylose, sodium hydrogen carbonate, amylopectin, sodium polyphosphate, and aluminium magnesium silicate.

The microneedle particles 14 may have a shape in which a diameter or area of a lower portion is wider than a diameter or area of an upper portion. That is, the microneedle particles 14 may be prepared in a shape of a cone or pyramid with a pointed upper end, or in a shape of having a tip at the top. As such, when the microneedle particles 14 are prepared in a form having a tip at the top, the microneedle particles 14 may penetrate the human skin more effectively when mixed with cosmetics or the like as described below.

For example, the microneedle particles 14 may be inserted into the skin and may dissolve, and may include a material that is biocompatible and biodegradable.

As examples thereof, hyaluronic acid and salts thereof, polyvinylpyrrolidone, polyvinyl alcohol, cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibrillatedcellulose, psyllium seed gum, xanthan gum, arabino galactan, gum arabic, alginic acid, gelatin, gellan gum, carrageenan, karaya gum, curdlan, chitosan, chitin, tara gum, tamarind gum, tragacanth gum, furcelleran, pectin or pullulan, hydroxypropyl methylcellulose, hydroxyalkyl cellulose, ethyl hydroxyethyl cellulose, alkyl cellulose, and carboxymethyl cellulose may be used.

The particle assembly 100 described above may be prepared using various known methods including a mold method. Therefore, the method of preparing the particle assembly 100 is not particularly limited.

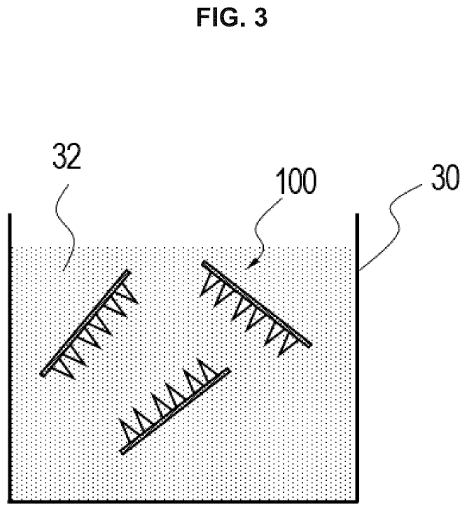

FIG. 3 is a conceptual diagram illustrating a process of treating the particle assembly 100 described above with a solvent 32. For example, FIG. 3 illustrates a process of treating the particle assembly 100 by using the liquid solvent 32.

Referring to FIGS. 2 and 3, the separating of the microneedle particles 14 may include weakening the adhesion between the first film 12 and the microneedle particles 14.

For example, the weakening of the adhesion between the first film 12 and the microneedle particles 14 may include dissolving the first film 12 or swelling the first film 12. First, a method of swelling the first film 12 is described below.

In the present embodiment, the particle assembly 100 described above may be treated with a predetermined solvent 32 such that the first film 12 of the particle assembly 100 swells with the solvent 32, thereby weakening the adhesion between the first film 12 and the microneedle particles 14.

In this case, the solvent 32 may be selected as a material that swells the first film 12 and does not dissolve the microneedle particles 14.

For example, the solvent 32 may include one or more substances selected from distilled water, ethanol, acetone, oleyl alcohol, isoparaffin, and dipropylene glycol. The solvent described above has a property by which the first film 12 swells well but the microneedle particles 14 including the component or material described above are not dissolved.

However, when the solvent 32 includes an ethanol aqueous solution and a concentration of ethanol in the ethanol aqueous solution is less than about 70% (i.e., a concentration of water is greater than about 30%), the microneedle particles 14 may be dissolved in the solvent 32. Therefore, a concentration of ethanol in the ethanol aqueous solution may be greater than or equal to 70%.

Accordingly, as shown in FIG. 3, the particle assembly 100 may be supplied to a housing 30 or tank containing a predetermined liquid solvent 32 and immersed in the liquid solvent 32. The particle assembly 100 is immersed in the solvent 32 and treated by the solvent 32.

In this case, as described above, the first film 12 of the particle assembly 100 may be swollen by the solvent 32.

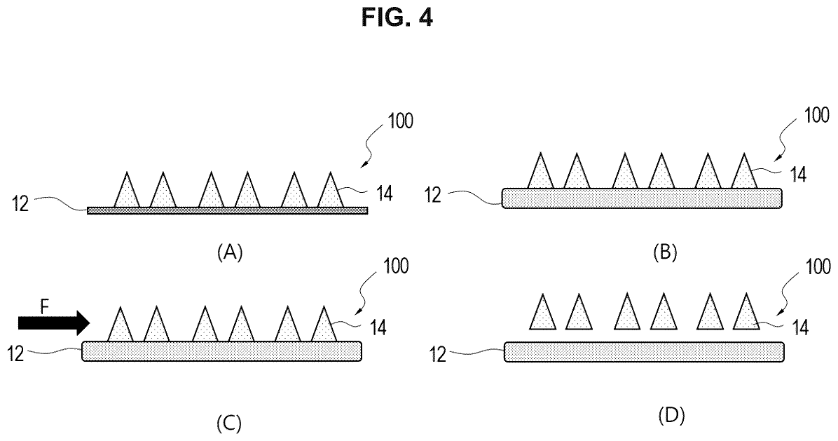

FIG. 4 is a diagram illustrating a process of separating microneedle particles 14 from the particle assembly 100 after solvent treatment.

(A) of FIG. 4 illustrates the particle assembly 100 before solvent treatment, and (B) of FIG. 4 illustrates the particle assembly 100 after solvent treatment.

The particle assembly 100 before solvent treatment as shown in (A) of FIG. 4 becomes swollen and inflated as shown in (B) of FIG. 4 after solvent treatment. When the first film 12 swells, a surface area of the first film 12 increases, and the adhesion between the microneedle particles 14 and the first film 12 becomes weaker than before the first film 12 swells.

As described above, the method may further include applying a transverse force to the microneedle particles 14 to separate the microneedle particles 14 from the first film 12 subsequently to the weakening of the adhesion between the first film 12 and the microneedle particles 14.

When a transverse force F is applied to the microneedle particles 14 as shown in (C) of FIG. 4, the microneedle particles 14 may be easily separated from the first film 12, and the microneedle particles 14 may be separated and extracted from the first film 12 as shown in (D) of FIG. 4.

The transverse force F applied to the microneedle particles 14 may be applied manually by a worker or automatically by a mechanical device.

After extracting microneedle particles by the method described above, the extracted microneedle particles are dried.

In the end, the microneedle particles 14, which is prepared by the method described above, may be provided in cosmetics including lotion and cream.

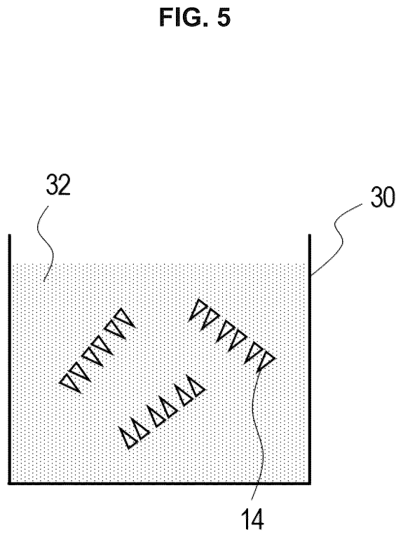

FIG. 5 is a conceptual diagram illustrating a process of separating microneedle particles 14 from the particle assembly 100 by dissolving the first film 12 in the weakening of the adhesion between the first film 12 and the microneedle particles 14.

Referring to FIG. 5, the separating of the microneedle particles includes separating the microneedle particles 14 from the film support layer 20 by treating the particle assembly 100 with the solvent 32 that dissolves the first film 12 but does not dissolve the microneedle particles 14.

That is, in the present embodiment, the first film 12 is dissolved by a predetermined solvent 32 to separate the microneedle particles 14 from the film support layer 20. In this case, the solvent 32 may be selected as a material that dissolves only the first film 12 and does not dissolve the microneedle particles 14.

In this case, the first film 12 of the particle assembly 100 is dissolved by the solvent 32, and thus the microneedle particles 14 remain in the solvent 32 as shown in FIG. 5.

Then, the microneedle particles 14 are filtered and extracted from the solvent 32, and the microneedle particles 14 are dried.

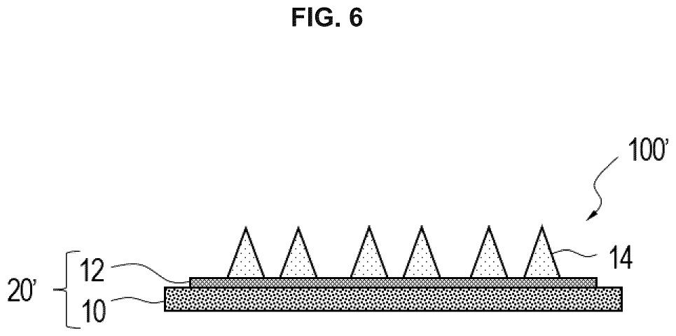

FIG. 6 is a diagram illustrating a particle assembly 100′ according to another embodiment of the present disclosure.

Referring to FIG. 6, the particle assembly 100′ according to the present embodiment may further include a second film 10 in a film support layer 20′ described above.

In this case, the second film 10 may be provided on a lower surface of the first film 12 described above. That is, the first film 12 may be formed on an upper surface of the second film 10 by using a coating method or the like.

The second film 10 may serve a support sheet or support film of the particle assembly 100.

The second film 10 may include a hydrophobic film, but is not limited thereto, and may also include a hydrophilic film.

For example, the second film 10 may include one or a mixture of two or more selected from high density polyethylene (HDPE), polystyrene (PS), polypropylene (PP), polyethylene terephthalate (PET), and polyethylene naphthalate (PEN).

The first film 12 may be selected such that the adhesion between the first film 12 and the microneedle particles 14 is higher than the adhesion between the second film 10 and the microneedle particles 14. The adhesion between the first film 12 and the microneedle particles 14 is described in detail in the preparing of the microneedle particles 14.

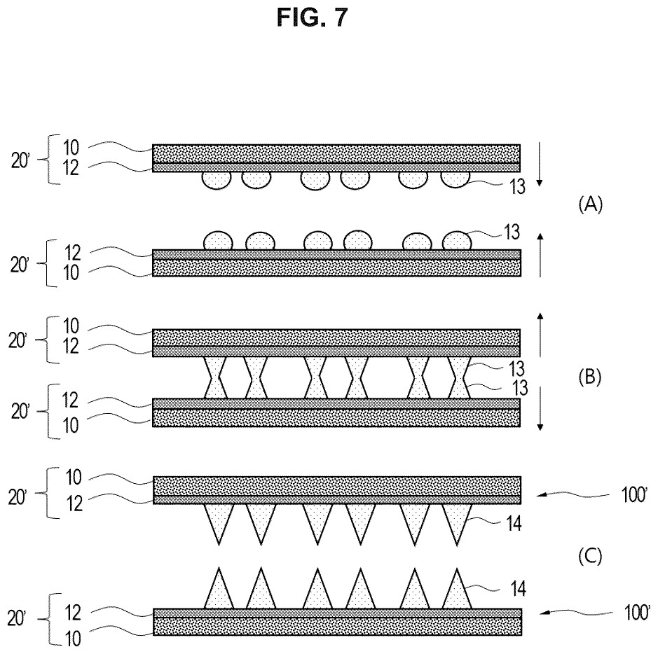

FIG. 7 is a diagram an operation of preparing the particle assembly 100′ according to another embodiment described above.

Referring to FIG. 7, the providing of the particle assembly 100′ may include providing a pair of the second films 10, providing the first film 12 formed on each of the pair of second films 10 to increase adhesion with the microneedle particles 14 and forming a pair of film support layers 20′, and spotting a biodegradable viscous material 13 on at least one of the first films 12 of the pair of film support layers 20′.

The pair of second films 10 may serve a support film of the film support layer 20′ of the particle assembly 100′.

The second films 10 may be provided, for example, on a pair of substrates (not shown). In this case, the second film 10 may be provided by being applied and dried on the substrate or may be provided in a sheet state.

The first film 12 may be provided on top of each of the pair of second films 10. The first film 12 may be applied onto the second film 10, thereby forming a film layer.

When the first film 12 is omitted, a viscous material 13 forming the microneedle particles 14 is spotted directly onto the second film 10. In this case, the adhesion between the viscous material 13 and the second film 10 is weak, and thus the viscous material 13 may not form a tip portion in a tensioning operation described below. That is, the viscous material 13 may be separated from the second film 10 during the tensioning operation, and thus the film support layer 20′ may not be sufficiently separated, and upper and lower viscous materials 13 are attached to each other to form microneedle particles. These microneedle particles have a so-called hourglass shape, which reduces their skin penetration effect.

Therefore, in the case of the present embodiment, the first film 12 is provided to the second film 10. In this case, the first film 12 may be selected such that the adhesion between the first film 12 and the microneedle particles 14 is higher than the adhesion between the second film 10 and the microneedle particles 14.

The adhesion between the first film 12 and the microneedle particles 14 may be determined to a level or higher that enables the viscous material 13 to form a tip portion when the viscous material 13 is tensioned by separating the pair of film support layers 20′ from each other in the tensioning operation described below.

(A) of FIG. 7 illustrates that the viscous material 13 is spotted on both the first films 12 of the pair of film support layers 20′ but the present disclosure is not limited thereto. For example, the viscous material 13 may be spotted on only one of the first films 12 of the pair of film support layers 20′.

Then, the pair of film support layers 20′ are moved relative to each other ((A) of FIG. 7) to come closer to each other, and thus the viscous material 13 and the first film 12 come into contact with each other between the first films 12 of the pair of film support layers 20′, and the pair of film support layers 20′ are separated from each other such that the viscous material 13 is tensioned ((B) of FIG. 7).

In this case, as described above, the adhesion between the viscous material 13 and the first film 12 may be determined to a level or higher that enables the viscous material 13 to form a tip portion.

Then, the viscous material 13 is solidified and the pair of film support layers 20′ are separated to form microneedle particles 14 on each of the first films 12 of the pair of film support layers 20′.

In this case, the viscous material 13 described above may be solidified using a method of blowing air or the like. When the viscous material 13 is sufficiently solidified and then the film support layers 20′ are further separated from each other, the microneedle particles 14 having a tip while the viscous materials 13 that are connected to each other are separated from each other ((C) of FIG. 7).

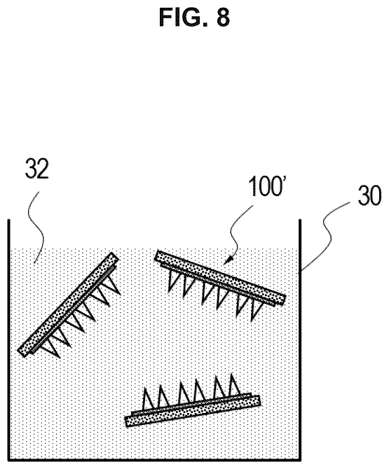

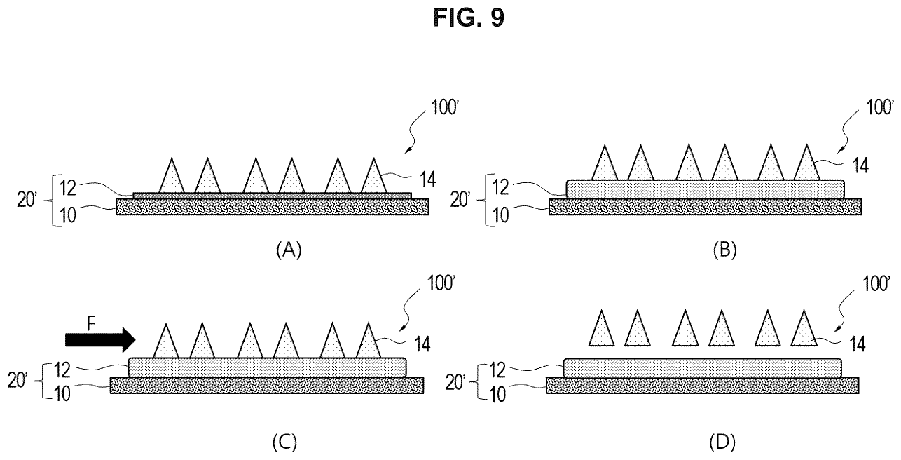

Then, the particle assembly 100′ is treated with the liquid solvent 32 as shown in FIG. 8, and then the microneedle particles 14 are separated from the particle assembly 100′ as shown in FIG. 9.

The particle assembly 100′ before solvent treatment as shown in (A) of FIG. 9 becomes swollen and inflated as shown in (B) of FIG. 9 after solvent treatment. When the first film 12 swells, a surface area of the first film 12 increases, and the adhesion between the microneedle particles 14 and the first film 12 becomes weaker than before the first film 12 swells.

Then, when a transverse force F is applied to the microneedle particles 14 as shown in (C) of FIG. 9, the microneedle particles 14 may be easily separated from the first film 12, and the microneedle particles 14 may be separated and extracted from the first film 12 as shown in (D) of FIG. 9.

The transverse force F applied to the microneedle particles 14 may be applied manually by a worker or automatically by a mechanical device.

After extracting microneedle particles by the method described above, the extracted microneedle particles are dried.

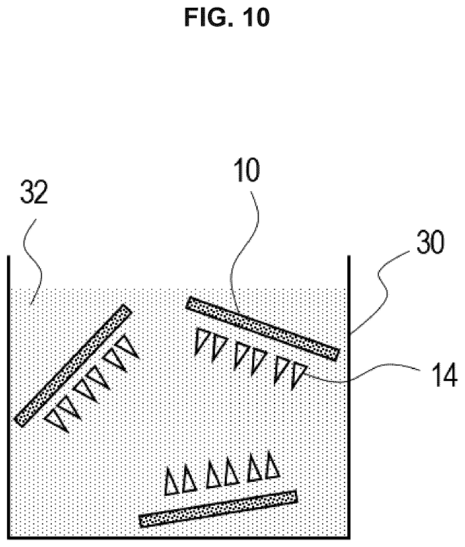

FIG. 10 is a conceptual diagram illustrating a process of separating microneedle particles 14 from the particle assembly 100′ by dissolving the first film 12 in the particle assembly 100′, according to another embodiment.

That is, the particle assembly 100′ is treated with the solvent 32 that dissolves the first film 12 but does not dissolve the microneedle particles 14. Accordingly, the microneedle particles 14 may be separated from the film support layer 20.

In this case, the first film 12 of the particle assembly 100′ is dissolved by the solvent 32, and thus the microneedle particles 14 are separated from the second film 10 and remain in the solvent 32 as shown in FIG. 10.

After the microneedle particles 14 and the second film 10 are separated from each other, the second film 10 is removed from the solvent 32 and the microneedle particles 14 are filtered and extracted. The second film 10 may remain in the solvent 32, and thus may be easily taken out and removed.

The microneedle particles 14 remain in the solvent 32, and thus the solvent 32 is filtered to extract the microneedle particles 14. Then, the microneedle particles are dried.

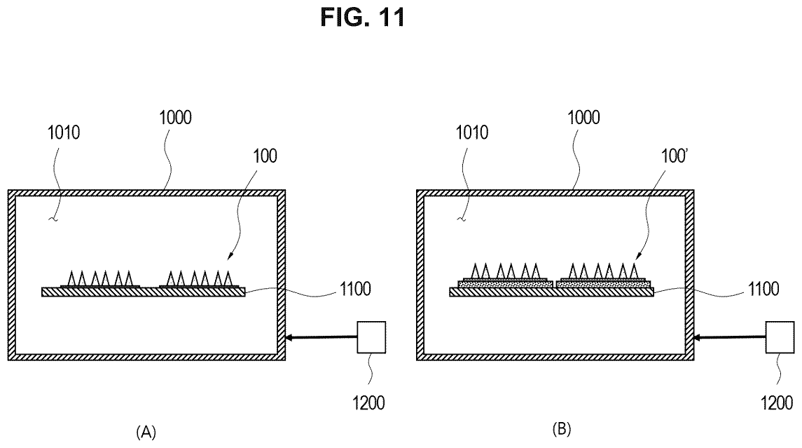

FIG. 11 is a schematic diagram illustrating a device for treating a particle assembly by vaporizing and providing a solvent. (A) of FIG. 11 illustrates a device for treating the particle assembly 100 with a gaseous solvent, according to an embodiment, and (B) of FIG. 11 illustrates a device for treating the particle assembly 100′ with a gaseous solvent, according to another embodiment.

Referring to FIG. 11, to treat the particle assemblies 100 and 100′ by using a vaporized solvent or a gaseous solvent, a sealed space for accommodating the particle assemblies 100 and 100′ is required.

In FIG. 11, a chamber 1000 is used to provide an accommodation space 1010 for accommodating the particle assemblies 100 and 100′ inside the chamber 1000. Although illustrated in the drawing as the chamber 1000, the present disclosure is not limited thereto and any sealed structure for accommodating the vaporized solvent and the particle assemblies 100 and 100′ may be used.

Although not shown in the drawing, the chamber 1000 may include an opening (not shown) through which the particle assemblies 100 and 100′ are to be placed into the accommodation space 1010 and a door (not shown) that opens or closes the opening.

A support plate 1100 on which the particle assemblies 100 and 100′ are mounted may be provided in the accommodation space 1010 of the chamber 1000. The support plate 1100 may be implemented with various structures on which the particle assemblies 100 and 100′ are accommodated or which supports the particle assemblies 100 and 100′.

A solvent supply unit 1200 for supplying vaporized solvent or a gaseous solvent into the chamber 1000 may be provided at one side of the chamber 1000.

For example, the solvent supply unit 1200 may vaporize ethanol and supply the same into the chamber 1000. The supply amount, supply time, and the like of the vaporized solvent by the solvent supply unit 1200 may be appropriately adjusted.

When the particle assemblies 100 and 100′ are treated with a vaporized solvent as illustrated in FIG. 11, the first film 12 described above in the particle assemblies 100 and 100′ is dissolved or swelled, thereby weakening the adhesion between the first film 12 and the microneedle particles 140 and separating the first film 12 and the microneedle particles 140, which is described above, and thus a repeated explanation is omitted.

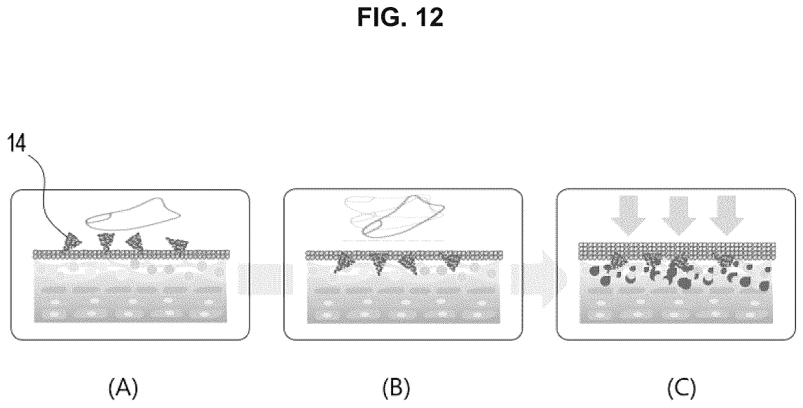

FIG. 12 is a conceptual diagram illustrating a process of using cosmetics containing microneedle particles 14.

When a user rubs the cosmetic against the skin as shown in (A) of FIG. 12, the microneedle particles 14 contained in the cosmetic may penetrate the skin as shown in (B) of FIG. 12.

In particular, the microneedle particles 14 according to the present embodiment are prepared in a form having a tip, and thus the skin penetration effect is excellent. The microneedle particles 14 include a material that is biocompatible and biodegradable, and thus are not harmful to the human body even when the microneedle particles 14 penetrate the skin.

As such, when microneedle particles 14 penetrate the skin, cosmetic ingredients may also penetrate the skin together with or along with the microneedle particles 14, as shown in (C) of FIG. 12.

The microneedle particles 14 may also include a functional material. For example, in the preparing of the particle assembly 100 described above, a functional material may be provided together with the viscous material forming the microneedle particles 14.

The functional material may include, for example, a wrinkle-improving material, a moisture-providing material, or the like and are not limited to a specific material in the present disclosure.

Although the present disclosure has been described above with reference to exemplary embodiments, those skilled in the art may modify and change the present disclosure in various ways without departing from the spirit and scope of the present disclosure as set forth in the claims described below. Therefore, when the modified implementation basically includes the elements of the claims of the present disclosure, it should be considered to be included in the technical scope of the present disclosure.

1. A method of preparing microneedle particles, the method comprising:
providing a particle assembly having a plurality of microneedle particles on an upper surface of a first film; and
separating the microneedle particles from the particle assembly,
wherein the separating of the microneedle particles from the particle assembly includes weakening adhesion between the first film and the microneedle particles.

2. The method of claim 1, wherein the first film includes a water-soluble film.

3. The method of claim 2, wherein the first film includes one or a mixture of two or more selected from polyvinylpyrrolidone, polyvinyl alcohol, cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibrillatedcellulose, psyllium seed gum, xanthan gum, arabino galactan, gum arabic, alginic acid, gelatin, gellan gum, carrageenan, karaya gum, curdlan, chitosan, chitin, tara gum, tamarind gum, tragacanth gum, furcelleran, pectin or pullulan, hydroxypropyl methylcellulose (HPMC), hydroxyalkyl cellulose, ethyl hydroxyethyl cellulose, alkyl cellulose, gluten, soy polysaccharides, polacrilin potassium, sodium starch glycolate, crospovidone, croscarmellose sodium, bentonite, hydroxypropyl starch, sodium carboxymethyl cellulose, sodium alginate, sodium lauryl sulfate, silicic anhydride, hydroxypropyl cellulose, polyvinyl acetate, amylose, sodium hydrogen carbonate, amylopectin, sodium polyphosphate, and aluminium magnesium silicate.

4. The method of claim 1, wherein the particle assembly further includes a second film attached to a lower surface of the first film.

5. The method of claim 4, wherein the second film includes a hydrophobic film or a hydrophilic film.

6. The method of claim 4, wherein the second film includes one or a mixture of two or more selected from high density polyethylene (HDPE), polystyrene (PS), polypropylene (PP), polyethylene terephthalate (PET), and polyethylene naphthalate (PEN).

7. The method of claim 4, wherein adhesion between the first film and the microneedle particles is higher than adhesion between the second film and the microneedle particles.

8. The method of claim 1, wherein the weakening of the adhesion between the first film and the microneedle particles includes treating the particle assembly with a solvent to dissolve or swell the first film.

9. The method of claim 8, wherein the solvent does not dissolve the microneedle particles.

10. The method of claim 8, wherein the solvent includes one or more selected from distilled water, ethanol, acetone, oleyl alcohol, isoparaffin, and dipropylene glycol.

11. The method of claim 8, further comprising, when the first film is swelled with the solvent, applying a transverse force to the microneedle particles to separate the microneedle particles from the first film.

12. The method of claim 8, further comprising:
when the first film is dissolved with the solvent, filtering and extracting the microneedle particles from the solvent; and
drying the microneedle particles.

13. The method of claim 8, wherein, when the particle assembly is treated with the solvent, the particle assembly is treated using the solvent in a liquid state or the solvent is vaporized to treat the particle assembly with the solvent in a gaseous state.

14. Microneedle particles prepared using the method of preparing microneedle particles of claim 1.

15. The method of claim 4, wherein the weakening of the adhesion between the first film and the microneedle particles includes treating the particle assembly with a solvent to dissolve or swell the first film.
